(19) United States
(12) Patent Application Publication
(10) Pub. No.: US 2024/0115225 A1
(43) Pub. Date: Apr. 11, 2024

(54) Method of Generating Successive Image Recordings

(71) Applicant: Agfa NV, Mortsel (BE)

(72) Inventor: Walter Exelmans, Mortsel (BE)

(73) Assignee: Agfa NV, Mortsel (BE)

(21) Appl. No.: 18/546,375

(22) PCT Filed: Feb. 10, 2022
(86) PCT No.: PCT/EP2022/053187

§ 371 Date: Aug. 14, 2023

(30) Foreign Application Priority Data
Feb. 16, 2021 (EP) 21157297.9

(51) Int. Cl. A61B 6/00

(52) U.S. Cl. CPC A61B 6/5211; A61B 6/486; A61B 6/542

(57) ABSTRACT

A first visual image of a region of interest is generated by means of an optical camera at the time when a first radiation image is recorded, a second visual image is generated by means of an optical camera of said same region of interest, first and second visual images are compared, and a second radiation image is recorded when said first and second visual images are substantially identical.

### FIELD OF THE INVENTION

The present invention relates to digital radiography.

The invention more particularly relates to a method of generating successive radiographic images of the same object in substantially identical exposure conditions.

### BACKGROUND OF THE INVENTION

X-ray imaging is a non-invasive technique to capture medical images of patients or animals.

Image recording comprises irradiating an object with an X-ray beam. As the X-ray beam passes through the object, it is attenuated by a degree that varies with the internal composition and/or thickness of the object. An X-ray detector is then arranged to capture the image-wise modulated X-ray beam and to convert the image-wise modulated X-ray beam into an image showing the internal structure of the object.

In the field of radiographic image recording it is nowadays common to use re-usable radiography detectors (also called ‘panels’, ‘flat panels’, ‘flat panel detectors’ etc.) for X-ray image recording.

Different types of such re-usable detectors exist.

One type of X-ray recording devices used in so-called computed radiography (CR) comprises a photo-stimulable phosphor detector which stores image-wise modulated radiation when it is exposed to an X-ray image.

To release the stored image, the photostimulable phosphor detector is scanned by means of stimulating radiation, e.g. by means of laser light of an appropriate wavelength adapted to the type photostimulable phosphor.

Upon stimulation the phosphor detector releases the stored radiation image in the form of image-wise modulated light which is then detected by a photo-electric convertor so as to generate an electric signal representation of the irradiated object.

This electric signal representation can then be processed in a signal processor and/or stored. It can further be applied to a display device or to a printer to generate a visible image on which an evaluation, e.g. a diagnosis in case of a medical image, can be performed.

Increasingly, radiography flat panel detectors (FPD) for so-called direct radiography (DR) are being used which can convert X-rays in a direct way (direct conversion direct radiography), or in an indirect way (indirect conversion direct radiography) into an electric signal representation.

In direct radiography, the radiography detectors convert X-rays directly into electric charges directly interacting with a photoconductive layer such as amorphous selenium (a-Se). In indirect conversion direct radiography, the radiography flat panel detectors have a scintillator layer consisting of CsI:Tl or Gd2O2S which converts X-rays into light which then interacts with the sensor being an amorphous silicon (a-Si) semiconductor layer, where electric charges are created.

In a hospital, more specifically in an intensive care unit, it is common practice to generate successive radiographic images of the same region of interest in order to be able to follow the health condition of a patient over time.

In order to be able to optimally compare successively taken radiographic images and to make observations and conclusions regarding the health condition of the patient, it is important that the successive radiographic images are generated under substantially identical exposure and image recording conditions.

A number of settings regarding exposure and image recording can be set identically at the operator's console when generating successive radiographic images, such as the amount of radiation that is emitted by a source of radiation. This amount of radiation is controlled by settings of the source of radiation and performed by the operator. Also collimator settings can be made identical during successive exposures.

However, the relative position of source of radiation and patient may change in an uncontrolled way in between successive exposures. For example, since the patient and/or the source of radiation can move in between successive exposures this can generate a change of relative position of patient and source of radiation and consequentially exposure and image recording conditions may not be identical during successive exposures and image recordings.

As a consequence the image interpretation needs to take this into account and becomes thus more complex or even not feasible.

It is an aspect of the present invention to provide a solution to the above-described problem.

### DESCRIPTION

The invention provides a method of generating successive image recordings of a patient by means of a source of penetrating radiation such as X-rays.

According to the invention, a first optical image of a region of interest is generated by means of an optical camera at the time when a first radiation image is recorded.

The region of interest may be a region part of the recording device the appearance of which may change in between two (successive) image recordings.

Examples of such regions of interest are the following:
The synchronisation can be controlled on the basis of the status of a resuscitation bag part of a respirator used for a patient. (Completely up or completely down).
Indicator LEDs of a display which give an indication on a monitored status of respiration or heart beat of a patient.
Still others may be envisaged.

Alternatively the region of interest may be located at the position of the patient.

For example an optical image of the patient's thorax may be taken so that the height of the thorax above the patient supporting table can be seen on the visual image.

Next, prior to the recording of a follow-up radiation image, a second visual image is generated by means of an optical camera (preferably the same optical camera) of the same region of interest.

Then, the first and the second visual images are compared.

This comparison can be performed manually by an operator inspecting the optical images when visualized on a display screen.

Alternatively, dedicated software may be used which compares the visual image taken at the time of the first recording with the images taken prior to the second recording.

Finally, a follow-up radiation image is recorded when said first and second visual images are found to be substantially identical.

1-3. (canceled)

4. A method of generating successive image recordings of a patient by means of a source of penetrating radiation, wherein
(i) a first visual image of a region of interest is generated by means of an optical camera at a time when a first radiation image is recorded,
(ii) a second visual image is generated by means of an optical camera of the region of interest,
(iii) the first visual image and the second visual image are compared, and
(iv) a second radiation image is recorded by the source of penetrating radiation when the first visual image and the second visual image are substantially identical.

5. The method of claim 4, wherein the region of interest is part of an x-ray exposure apparatus, the appearance of which may change between said first and second image recording.

6. The method of claim 4, wherein the region of interest is defined as a region of interest on the patient.

7. The method of claim 4, wherein the penetrating radiation is an X-ray.

8. The method of claim 5, wherein the penetrating radiation is an X-ray.

9. The method of claim 6, wherein the penetrating radiation is an X-ray.
